(19) United States
(12) Patent Application Publication
(10) Pub. No.: US 2018/0353333 A1
(43) Pub. Date: Dec. 13, 2018

(54) Film Including a Polysulfated Oligosaccharide and a Polycation and Method for Manufacturing Same

(71) Applicants: Urgo Recherche Innovation et Developpement, Chenove (FR); Centre National de la Recherche Scientifique-CNRS-, Paris (FR)

(72) Inventors: Fouzia Boulmedais, Strasbourg (FR); Pierre Schaaf, Molsheim (FR); Jean-Marc Pernot, Dijon (FR); Christelle Laurensou, Dijon (FR)

(73) Assignees: Urgo Recherche Innovation et Developpement, Chenove (FR); Centre National de la Recherche Scientifique -CNRS -, Paris (FR)

(21) Appl. No.: 15/781,817

(22) PCT Filed: Dec. 2, 2016
(86) PCT No.: PCT/FR2016/053191

§ 371 Date: Jun. 6, 2018

(30) Foreign Application Priority Data
Dec. 7, 2015 (FR) 15 61928

(51) Int. Cl. A61F 13/00; C08L 5/00; C08L 77/04; C08L 79/02; C08K 5/41

(52) U.S. Cl. CPC A61F 13/00012; C08L 5/00; C08L 77/04; C08G 73/0206; C08K 5/41; C08L 2203/02; C08L 79/02

(57) ABSTRACT

The present invention relates to a nanofilm including at least one polycation and one polysulfated oligosaccharide. The present invention also relates to the nanofilms obtainable by simultaneously or alternately sputtering at least one polycation solution and at least one polysulfated oligosaccharide solution and to the use of said nanofilms on skin, skin conditions, wounds, or mucous membranes. The present invention also relates to a nanofilm for the use thereof in a method for releasing polysulfated oligosaccharide upon contact with exuding wounds.

The present invention aims for a nanofilm including at least one polycation and one agent. This agent is chosen among synthetic polysulfated oligosaccharides having 1 to 4 ose units, their salts, or their complexes. The present invention is also based on nanofilms obtainable by simultaneously or alternately sputtering, of at least one polycation solution and at least one polysulfated oligosaccharide solution, and to the use of these nanofilms on skin, skin conditions, wounds or mucous membranes. The present invention is also based on a nanofilm for the use thereof in a method for releasing polysulfated oligosaccharide upon contact with exuding wounds.

The healing of a wound is a natural biological process, human and animal tissues being capable of repairing localised lesions by repair and regeneration processes which are specific to them.

The speed and quality of healing of a wound depends on the general state of the organism damaged, the aetiology of the wound, the state and the localisation of the wound, and the occurrence or not of an infection, as well as genetic factors predisposing or not problems with healing.

The natural healing of a wound mainly takes place according to three successive phases, each one of these phases being characterised by specific cellular activities which make the repair process progress according to specific chronological sequences: the inflammatory phase, the granulation phase (or proliferative phase), and the scar forming phase.

The first phase, the inflammatory phase, starts from the rupture of blood vessels which triggers the formation of a clot (blood coagulation) mainly composed of fibrine and fibronectin, and which will constitute a provisional matrix. This matrix, in part, fills the lesion and will enable the migration within the damaged zone of the inflammatory cells recruited to ensure the detersive cleaning of the wound. The platelets present will also release factors (for example, cytokine, growth factors) enabling the recruitment of healing cells as they will inflammatory cells (polynuclear neutrophils and macrophages), fibroblasts and endothelial cells.

The second phase corresponds to the development of granulation tissue. First, a colonisation of the injury by proliferating fibroblasts is observed. Then, the migration of the endothelial cells from the blood vessels will enable neovascularisation, or angiogenesis, of the damaged tissue. In the granulation tissue, the fibroblasts are activated and will vary in myofibroblasts having significant contractile properties, generated by actin microfilaments, enabling the contraction of the wound. These microfilaments are expressed by a protein: the α-muscular actin smoothens. These myofibroblasts therefore play a major role in the formation and maturation of the granulation tissue which will lead to the healing of the lesion. There, there is migration of the reconstruction keratinocytes of the epidermis.

The third phase of the repair process, the formation of the scar or maturation, is accompanied by a remodelling of the granulation tissue. A part of the extracellular matrix is directed by proteinases (mainly matrix metalloproteinases (MMP) and elastases), and a progressive reorganisation of the extracellular matrix is observed. Progressively, type III collagen, dominant in the granulation tissue, is replaced by type I collagen, main matrix component of the dermis. At the end of the maturation phase, the fibroblasts, myofibroblasts and vascular cells become proliferated and/or have their activity reduced. Then, the surplus cells die through apoptosis. Alongside the remodelling of the extracellular matrix and the apoptosis of the surplus cells, the inflammatory state progressively decreases. This phase is longer: at the end of around one year, the scar is remodelled, it is no longer red, nor rigid, and no longer causes pain and it is flattened.

Polysulfated oligosaccharides are known and used to treat various skin pathologies, and particularly for treatment of wounds. As an example, products from the “Cicalfate” range can be cited, commercialised by Laboratoires Pierre Fabre to favour the repair of the epidermis, which contain a polysulfated oligosaccharide, sucralfate. These products are presented in the form of cream.

“UrgoStart” products can also be cited, commercialised by Laboratoires Urgo, for the treatment of wounds which contain a polysulfated oligosaccharide, potassium sucrose octasulfate. These products are presented in the form of dressings. The potassium sucrose octasulfate is incorporated to the lipidocolloid matrix.

The present invention is based on a new mode of incorporating polysulfated oligosaccharides in the form of nanofilms enabling their release when they are in contact with skin, wounds or mucous membranes.

In particular, the invention aims for a nanofilm, characterised in that it includes at least one polycation and one polysulfated oligosaccharide having one to four ose units.

The nanofilms of the invention are obtainable by sputtering at least one polycation solution and at least one polysulfated oligosaccharide solution.

It is understood, by nanofilm, in the sense of the present invention, films having a thickness of between 1 nm and 10 μm, preferably less than 10 μm. According to a preferred embodiment, the nanofilms of the invention have a thickness of 10 nm to 8 μm, more preferably from 50 nm to 5 μm, more preferably still from 100 nm to 1 μm.

The nanofilm according to the invention is characterised in that it includes at least one polycation and at least one polysulfated oligosaccharide.

The nanofilms according to the present invention are obtainable by simultaneously or alternately sputtering, of at least one polycation solution and at least one polysulfated oligosaccharide solution.

The nanofilms of the invention are particularly useful when they are applied on a wound. Indeed, upon contact with exudates, the nanofilm is quickly dissolved, enabling the release of the polysulfated oligosaccharides. This can enable an immediate action upon contact with the exudates and therefore reach the effective dose more quickly. In addition, the nanofilms of the invention contain an optimal quantity of polysulfated oligosaccharide for the treatment of wounds, while limiting the quantities of raw materials used in their manufacturing method.

The present invention also aims for a nanofilm for the use thereof in a method for releasing polysulfated oligosaccharide upon contact with exuding wounds, said method including, in particular, a first step of preparing a nanofilm according to the invention, and a second step of putting the nanofilm in contact with an exuding wound.

Thus, the present invention is more specifically concerned about the use of nanofilms containing the polycation and polysulfated oligosaccharides for the treatment of exuding wounds. Indeed, in the presence of exudates, the nanofilms dissolve and release the agent that they contain.

Polysulfated Oligosaccharides

The oligosaccharides used in the framework of the present invention are synthetic oligomers formed of 1 to 4 ose units, and preferably, 1 or 2 ose units, generally connected to each other by alpha or beta glycosidic connection. In other words, mono, di, tri or tetrasaccharides, and preferably mono or disaccharides.

There is no specific limitation affecting the type of ose units of these polysaccharides. Preferably, they will be pentoses or hexoses. As an example of monosaccharide, glucose, galactose or mannose can be cited. As an example of disaccharide, maltose, lactose, sucrose or trehalose can be cited. As an example of trisaccharide, melezitose can be cited. As an example of tetrasaccharide, stachyose can be cited.

Preferably, the oligosaccharide is a disaccharide, and preferably still, sucrose. It is understood by “polysulfated oligosaccharide” in the sense of the present application, an oligosaccharide of which at least two, and preferably all hydroxyl groups of each ose have been substituted by a sulphate group.

Preferably, the polysulfated oligosaccharide used in the framework of the present application is sucrose octasulfate.

The polysulfated oligosaccharides used in the framework of the present invention can be presented in the form of salts or complexes.

As an example of salts, alkaline metal salts can be cited, such as sodium, calcium or potassium salts; silver salts; or amino acid salts.

As an example of complexes, hydroxy-aluminium complexes can be cited.

In the framework of the present invention, particularly preferred compounds are as follows:
octasulfated potassium sucrose salt (or potassium sucrose octasulfate);
octasulfated silver sucrose salt; and
the hydroxyl-aluminium complex of sucrose octasulfate, commonly called sucralfate.

Potassium sucrose octasulfate (or KSOS) is particularly preferred. It is presented in the form of a multicharged molecule which has Matrix Metalloproteinase inhibiting properties. It is presented in the form of a small, negatively charged polymeric chain (polyanion).

In the framework of the present invention, the polysulfated oligosaccharides are used in quantities going from 10% to 90%, preferably from 30% to 70%, by weight in relation to the weight of the nanofilm.

In particular, in the framework of the present invention, the polysulfated oligosaccharides used are preferably potassium salts.

The polysulfated oligosaccharides used in the framework of the present invention can be presented in the form of micronised powder or in dissolved form.

Potassium sucrose octasulfate salt has been known for 25 years for the treatment of wounds during the sprouting phase, thanks to the action thereof on fibroblasts. This action is, for example, defined in patent applications EP 230 023, WO 89/05645 or WO 98/22114. As is specified in these documents, this compound is used after having carried out an assisted detersive cleaning of the wound and therefore after having removed the necrotic and/or fibrinous tissues (see EP 230 023, WO 89/05645, WO 98/22114). Therefore, it is used on a clean and cleansed wound.

In the international patent application WO 89/05645, it is specified that polysulfated polysaccharides such as sucralfate are proteinase inhibitors, particularly hyaluronidases, which are enzymes capable of damaging hyaluronic acid and glycosaminoglycans.

One of the current developments of biomaterials is oriented towards the production of smart bioagent materials. Different surface treatment techniques have been developed with this aim.

Polycation

A way of functionalisation, based on the layer-by-layer deposit of polyelectrolytes (alternatively a polycation and a polyanion), has been proposed at the start of the 1990s.

The thickness of the nanofilms obtained can be controlled by making several parameters vary (number of layers, molecular masses, concentrations and type of polyelectrolytes, pH level, ionic force). Their interest falls under the field of biomaterials by the possibility of giving them a biological functionality by inserting proteins, peptides or medication. The multilayer films are obtained mainly by two techniques: by alternate impregnations in polyelectrolyte solutions or by the alternate or simultaneous sputtering of these solutions. The cohesion of these films is based mainly on the electrostatic interactions between the positive and negative charges of the polycation and polyanion chains, but also between polyelectrolytes and small multicharged molecules.

In the framework of the present invention, the nanofilm includes at least one polycation, in other words, a positively-charged ionic polymer.

The nanofilms of the invention are obtained by combining polysulfated oligosaccharide with large chain polycations.

Preferably, the polycations implemented can be chosen among poly(L-lysin), chitosan, collagen, branched poly(ethylene imine), poly(L-arginine), poly(allylamine) (PAH), and their mixtures; in particular, poly(L-lysin) and branched poly(ethylene imine).

The cohesion of these nanofilms is based on electrostatic interactions between the positive charges of the polycation chains and the negative charges of the polysulfated oligosaccharide polyanion.

These polycations are preferably present in the nanofilms of the invention in quantities going from 10% to 90%, preferably from 30% to 70% by weight in relation to the weight of the nanofilm.

Pharmaceutically-Acceptable Environment

The nanofilm of the invention is particularly obtainable by simultaneously or alternately sputtering (or spraying), or at least one polycation solution and at least one polysulfated oligosaccharide solution. In particular, the nanofilm can be obtained by a succession of alternating sputtering, several times, the sputtering of a polycation solution and the sputtering of a polysulfated oligosaccharide solution.

The polycation and the polysulfated oligosaccharide are such as defined above.

The nanofilm of the invention is particularly obtainable by 1 to 500 steps of sputtering the polycation solution and the polysulfated oligosaccharide solution, the two solutions being simultaneously or alternately sputtered, preferably simultaneously.

Indeed, the simultaneous sputtering of the polycation and polysulfated oligosaccharide solutions can particularly enable to achieve the desired thickness with a reduced number of steps in relation to alternate sputtering. In addition, the thickness of the nanofilm obtained by simultaneous sputtering is easier to control so that it can be reproduced, compared with alternate sputtering. Thus, alternate sputtering enables to optimise the quantity of polysulfated oligosaccharide inserted in the nanofilm.

Preferably, the nanofilm is obtained by 5 to 200 steps of sputtering, and more preferably, by 20 to 100 steps of sputtering the polycation solution and the polysulfated oligosaccharide solution.

The liquid solutions including the polycation or polysulfated oligosaccharide particularly include a pharmaceutically-acceptable environment which could contain water.

By pharmaceutically-acceptable environment, this means, in the sense of the present application, an environment that is compatible with polycations and/or polysulfated oligosaccharides.

Water can particularly be present in liquid solutions including the polycation or polysulfated oligosaccharide in a content higher than 70%, preferably higher than 95%.

In particular, liquid solutions including the polycation or polysulfated oligosaccharide can include a mixture of water (i.e. as a pharmaceutically-acceptable environment) and sodium chloride (NaCl), “substantive salt”, preferably at a concentration of 150 mM.

Each sputtering step can be carried out for a duration going from 1 second to 1 minute, preferably from 5 seconds to 10 seconds.

The sputtering flow can, for example, be between 0.1 mL/s and 1 mL/s, preferably between 0.2 mL/s and 0.5 mL/s.

Each sputtering step is preferably followed by a rinsing step and/or a drying step.

Each rinsing step and each drying step is preferably carried out for a duration going from 1 second to 1 minute, preferably from 5 seconds to 10 seconds.

The nanofilm of the invention can also be obtained by a method of alternate impregnation (rolling/dipping) in at least one polycation solution and at least one polysulfated oligosaccharide solution.

The polycation and polysulfated oligosaccharide are such as defined above.

The nanofilm of the invention is particularly obtainable by 1 to 500 alternate impregnation steps in the polycation solution and the polysulfated oligosaccharide solution. Preferably, the nanofilm is obtained by 5 to 200 alternate impregnation steps, and more preferably, by 20 to 100 alternate impregnation steps in the polycation solution and the polysulfated oligosaccharide solution.

Liquid solutions including the polycation or polysulfated oligosaccharide particularly include a pharmaceutically-acceptable environment which could contain water.

Water can particularly be present in liquid solutions including the polycation or polysulfated oligosaccharide in a content higher than 70%, preferably higher than 95%.

In particular, liquid solutions including the polycation or polysulfated oligosaccharide can include a mixture of water (i.e. as a pharmaceutically-acceptable environment) and sodium chloride (NaCl), “substantive salt”, preferably at a concentration of 150 mM.

Each impregnation step can be carried out for a duration going from 1 minute to 15 minutes, preferably from 5 minutes to 10 minutes.

Each impregnation step is preferably followed by a rinsing step and/or a drying step.

Each rinsing step is preferably carried out for a duration going from 1 minute to 15 minutes, preferably from 5 minutes to 10 minutes.

Each drying step is preferably carried out for a duration going from 1 second to 1 minute, preferably from 5 seconds to 10 seconds.

The invention also aims to combine liquid compositions, characterised in that it includes at least one polycation solution and at least one polysulfated oligosaccharide solution defined above.

The invention is illustrated in more detail in the following non-limitative examples.

### EXAMPLES OF NANOFILMS ACCORDING TO THE INVENTION

The polycations which have been used are of synthetic origin, like poly(L-lysin) (polypeptide) or of natural origin, like collagen (COL, protein).

Potassium sucrose octasulfate (or KSOS) and the polycations have been prepared in ultra-pure water (with resistivity 18.2 MΩ·cm, Milli-Q-plus system, Millipore).

The construction of nanofilms has been done on silicon substrates (Wafernet INC, USA), cut beforehand using a diamond scraper into rectangles of around 2×2 cm². Before use, the substrates have been treated with plasma cleaner (Harrick Plasma, USA) in order to remove impurities and make them hydrophilic. The gas used is oxygen contained in air.

Whichever the system studied, a first layer of branched poly(ethylene imine) (BPEI) has been deposited by dipping (5 minutes) the substrate in an aqueous BPEI solution followed by two rinses with water (each for 1 minute) and a drying with nitrogen. This first layer serves as a fastening layer favouring the construction of nanofilms.

The preparation of polycation/KSOS nanofilms by sputtering is defined below. A sputtering device including four airbrushes, connected to a compressed air inlet and placed at a distance of 25 cm from the substrate has been used.

Two airbrushes enable to sputter the two compounds in solution (KSOS and the chosen polycation). Two others enable to carry out the steps of rinsing and drying the deposits.

In order to evaluate the quantity of sputtered compounds, the sputtering flows are measured during each handling and are around 0.3 mL/s.

The substrate is rotated for a duration of the sputtering around 1000 rpm in order to obtain consistent deposits.

Several parameters have been studied:
the type of sputtering (alternate or simultaneous)
the concentration of substantive salt NaCl (0 and 150 mM)
the type of polycations (synthetic or natural origin)
the sputtered KSOS/polycation ratio

In order to measure the thickness of successive deposits on the silicon substrates, ellipsometry has been used.

Nanofilm stability tests have been carried out by putting a substrate of 2×2 cm² in contract with 20 mL of physiological environment and environment simulating the wound for 45 minutes.

The physiological environment is modelled by a phosphate buffer at pH level 7.4±0.1 (phosphate buffer saline, PBS) obtained by diluting a 10× concentrated PBS solution containing Ca²⁺ and Mg′.

The wound environment is modelled by a 5% mass NaCl and 5% mass CaCl₂ dihydrate aqueous solution.

### Example 1 According to the Invention: Poly(L-Lysin)/KSOS Nanofilms

PLL/KSOS nanofilms have been obtained after 25 simultaneous sputtering steps. The sputtering durations are as follows: 10 seconds for sputtered compounds; 5 seconds for rinsing, 5 seconds for drying.

The two compounds are used in an aqueous solution with 150 mM NaCl in substantive salt.

The optimisation of the construction has been obtained by making the KSOS concentration vary from 0.05 to 12 mg/mL for a PLL concentration of 0.2 mg/mL.

The construction optimum is obtained with 0.2 mg/mL in KSOS.

After developing PLL/KSOS nanofilms, their stability has been tested upon contact with the physiological environment and upon contact with the wound environment. To do this, each nanofilm with a size of 2×2 cm² has been put in contact with 750 μL of physiological environment or wound environment for 45 minutes followed by a step of rinsing with Milli Q water for 45 minutes.

PLL/KSOS nanofilms have a low loss of thickness (<10%) upon contact with the physiological environment, which means that nanofilms are stable upon contact with the physiological environment. In addition, the infrared spectroscopy shows that a low portion of the KSOS is released during this contact: the 1240 cm⁻¹ strip of KSOS slightly decreases.

However, upon contact with the wound environment, PLL/KSOS nanofilms are totally dissolved, thus releasing all the KSOS.

### Example 2 According to the Invention: Study of Sputtering in Obtaining Poly(L-Lysin)/KSOS Nanofilms

In this example, the two compounds (PLL and KSOS) are used in an aqueous solution with 150 mM NaCl in substantive salt and at a concentration of 0.2 mg/mL.

PLL/KSOS nanofilms have been obtained after 16 alternate or simultaneous sputtering steps.

An alternate sputtering step is carried out as follows: the PLL solution then the KSOS solution is sputtered for 5 seconds followed by a step of rinsing for 5 seconds and of drying for 5 seconds.

A simultaneous sputtering step is carried out as follows: the PLL and KSOS solutions are simultaneously sputtered for 5 seconds, followed by a step of rinsing for 5 seconds and of drying for 5 seconds.

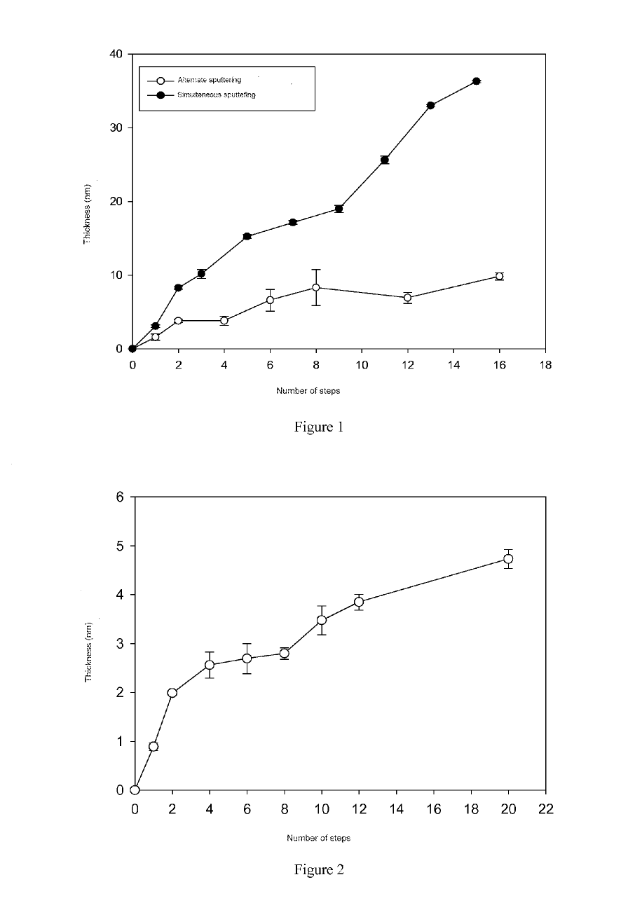

FIG. 1 represents the development of the thickness, in nm, of PLL/KSOS nanofilms according to the number of alternate and simultaneous sputtering steps in the presence of 150 mM NaCl. After 16 sputtering steps, the PLL/KSOS nanofilm reaches 36 nm by the simultaneous sputtering method and 10 nm by the alternate sputtering method.

The simultaneous sputtering method enables to obtain PLL/KSOS nanofilms with a given thickness with less steps than alternate sputtering. Indeed, all is needed is 3 simultaneous sputtering steps to obtain a 10 nm nanofilm, whereas 16 alternate sputtering steps are needed. Thus, the simultaneous sputtering method is economically advantageous (quicker and enables to use lower quantities of PLL and KSOS).

In addition, the thicknesses obtained in simultaneous sputtering have a better reproducibility (thickness error bar less than 10% thickness). Thus, the thickness of the nanofilm is easier to control with simultaneous sputtering.

In addition, the thicknesses obtained in alternate sputtering reach an upper value of 10 nm over the number of steps studied. This means that between 6 and 16 steps, the thickness no longer increases.

In conclusion, simultaneous sputtering enables a more effective control of the quantity of KSOS inserted in the nanofilm compared with alternate sputtering.

### Example 3 According to the Invention: Branched Poly(Ethylene Imine) (BPEI)/KSOS nanofilms obtained by alternate impregnation

The two compounds (BPEI and KSOS) are used in an aqueous solution with 150 mM NaCl in substantive salt and at a concentration of 0.2 mg/mL.

BPEI/KSOS nanofilms have been obtained after 20 alternate impregnation steps. An impregnation step is carried out as follows: the support is dipped in one of the solutions (BPEI or KSOS) for 5 minutes, followed by a step of rinsing (dipping in a 150 mM NaCl solution) for 5 minutes and of drying for 5 seconds.

FIG. 2 represents the development of the thickness, in nm, of BPEI/KSOS nanofilms according to the number of alternate impregnation steps in the presence of 150 mM NaCl. After 20 alternate impregnation steps, the BPEI/KSOS nanofilm reaches 4.7 nm. As a comparison, the thickness of BPEI/KSOS nanofilms reach around 15 nm by alternate and simultaneous sputtering. One of the advantages of the impregnation method is the volume used in a compound solution. Indeed, for 20 deposit steps, the volume used for each compound is 10 mL in alternate impregnation and 30 mL in alternate and simultaneous sputtering.

1. A film comprising at least one polycation and one polysulfated oligosaccharide having one to four ose units, said film having a thickness between 1 nm and 10 μm.

2. The film according to claim 1, wherein the polycation is present in a quantity of 10% to 90% by weight, and the polysulfated oligosaccharide is present in a quantity of 10% to 90% by weight in relation to the weight of the film.

3. The film according to claim 1, wherein the polycation is selected from the group consisting of poly(L-lysin), chitosan, collagen, branched poly(ethylene imine), poly(L-arginine), poly(allylamine) (PAH), and their mixtures.

4. The film according to claim 1, wherein the polysulfated oligosaccharide is a sucrose octasulfate.

5. The film according to claim 1, having a thickness of 10 nm to 8 μm.

6. A film obtained by simultaneously or alternately sputtering, of at least one polycation solution and at least one polysulfated oligosaccharide solution.

7. The film according to claim 6, wherein the sputtered solutions include a pharmaceutically-acceptable environment.

8. The film according to claim 6, wherein the sputtering is carried out simultaneously.

9. A film obtained by alternate impregnation in at least one polycation solution and at least one polysulfated oligosaccharide solution.

10. The film according to claim 9, wherein the solutions include a pharmaceutically-acceptable environment.

11. A method for releasing a polysulfated oligosaccharide onto an exuding wounds, said method comprising:
preparing a film according to claim 1, and contacting the film with an exuding wound to release the polysulfated oligosaccharide.

12. A device configured to protect skin, wounds or mucous membranes, said device comprising the film according to claim 1.

13. The film according to claim 2, wherein the polycation is present in a quantity of 30% to 70% by weight and the polysulfated oligosaccharide is present in a quantity of 30% to 70% by weight.

14. The film according to claim 3, wherein the polycation is poly(L-lysin) or branched poly(ethylene imine).

15. The film according to claim 4, wherein the sucrose octasulfate is potassium sucrose octasulfate.

16. The film according to claim 5, having a thickness of 50 nm to 5 μm.

17. The film according to claim 5, having a thickness of 100 nm to 1 μm.

18. The film according to claim 7, wherein the pharmaceutically-acceptable environment is water.
